(19) United States
(12) Patent Application Publication
(10) Pub. No.: US 2023/0092452 A1
(43) Pub. Date: Mar. 23, 2023

(54) TAPERED LOBULAR SURGICAL DRIVER AND IMPLANT SYSTEM AND TECHNIQUE FOR DISENGAGING DRIVER DURING SURGERY

(71) Applicant: Treace Medical Concepts, Inc., Ponte Vedra, FL (US)

(72) Inventors: Adam DeBosier, Jacksonville, FL (US); Michael Stedham, Jacksonville, FL (US); Robert Santrock, Morgantown, WV (US); William T. DeCarbo, Mars, PA (US); W. Bret Smith, Durango, CO (US); Daniel Hatch, Greeley, CO (US); Paul Dayton, Ankeny, IA (US); Jody McAleer, Jefferson City, MO (US)

(21) Appl. No.: 17/948,847

(22) Filed: Sep. 20, 2022

Related U.S. Application Data
(60) Provisional application No. 63/246,772, filed on Sep. 21, 2021.

(51) Int. Cl. A61B 17/88; A61B 17/86

(52) U.S. Cl. CPC A61B 17/8872; A61B 17/8888; A61B 17/8605

(57) ABSTRACT

A surgical technique can be performed utilizing a detachable driver-implant system and comparatively small surgical incision. The clinician can make an incision through the skin of the patient and retract the skin along the incision to expose a first bone region. The clinician can insert an implant into one or more bone portions using a detachable driver-implant system and then detach the driver, leaving the implant in the one or more bone portions. The clinician can reposition the skin of the patient at least partially over the end of the implant to expose a second bone region not exposed while the first bone region was exposed. After optionally performing one or more surgical steps on second bone region, the clinician can reposition the skin to again expose the first bone region. The clinician can then reattach the driver to the implant and remove the implant from the bone portions.

### CROSS-REFERENCE

This application claims the benefit of U.S. Provisional Patent Application No. 63/246,772, filed Sep. 21, 2021, the entire contents of which are incorporated herein by reference.

### TECHNICAL FIELD

This disclosure relates to orthopedic driver systems and surgical techniques utilizing an orthopedic driver.

### BACKGROUND

A variety of different orthopedic procedures involve fixation of two or more bones or bone portions relative to each other. For example, a clinician may insert pins, screws, staples, and/or other fixation elements into adjacent bones to hold the position of the bones relative to each other. The fixation may be temporary, e.g., by inserting a fixation element to hold a bone position while the clinician performs one or more other surgical steps with the fixation element then being removed prior to completion of the surgical procedure. Alternatively, the fixation may be permanent, e.g., by inserting a fixation element that remains in patient's body after completion of the surgical procedure.

In either case, a clinician may use a driver to install a fixation element into one or more bones or bone portions of a patient during a procedure. In some implementations, the driver includes a male engagement end that inserts into a female receiving end of the fixation element. The clinician attaches the driver to the fixation element, drives the fixation element into one or more bones, and then disengages the driver from the fixation element.

While precise placement of a fixation element into a bone is desired for all applications, precision targeting and delivery of a fixation element is particularly important when operating on comparatively small bones. Example small bone procedures include those performed on the hand or foot, where the bones are comparatively small compared to other areas of the anatomy. If a fixation element shifts or angles relative to an engagement end of a driver during insertion, the relative movement can cause the fixation element to be inserted into the bone off of a target trajectory. When working on small bones, minor errors in targeting trajectory may have a comparatively large effect on the efficacy of fixation because of the lack of bone volume to compensate for errant targeting.

In either case, during an orthopedic procedure involving insertion of a fixation element, the clinician may make one or more incisions through the patient's skin to access the underlying bones to perform the procedure. A longer incision provides the surgeon with greater access to perform the procedure. However, a longer incision results in a longer scar for the patient after healing, which can be cosmetically undesirable. For this reason, the patient may prefer a shorter incision and/or fewer incisions. This can be challenging for the surgeon because it limits access for performing the procedure.

### SUMMARY

In general, this disclosure is directed to orthopedic implants and driver systems, techniques for using such systems, and orthopedic procedures involving detachable implant-driver systems. In some examples, a surgical driver and implant system is described that includes a bone implant configured for insertion into mammalian (e.g., human) bone and a driver releasably attachable to the bone implant. The bone implant can have a socket providing a female receiving cavity, and the driver can have a male engagement portion that is insertable into the socket of the bone implant to engage the driver with the bone implant. The socket of the bone implant can have a tapered sidewall such that the cross-section or open area of the socket decreases moving from the outer end of the implant to the bottom of the socket. The engagement portion of the driver may have a corresponding taper that is configured mate with the socket of the implant (e.g., size and/or shape indexed to the size and/or shape of the socket). When the driver is inserted into the socket of the implant, the entire length of the driver inserted into the socket may contact a corresponding sidewall region of the socket.

In practice, configuring an engagement portion of a driver with a tapered engagement portion can be useful to help the clinician easily guide the driver into the corresponding socket of the bone implant. Further, by configuring the socket of the bone implant in the engagement portion of the driver to have corresponding taper profiles, the head of the driver can tightly engage with the interior wall surface of the bone implant socket, e.g., as the driver is inserted into the socket. This can help prevent angular shifting or canting of the bone implant relative to the driver, which may otherwise occur if a tapered driver is inserted into a straight walled socket with free space between the wall of the socket and the tapered surface of the driver. For high precision orthopedic applications, minor shifting between the driver and implant can change the trajectory of the implant as it is inserted into the bone.

In some configurations, the driver is configured with one or more lobes that are insertable into one or more corresponding grooves of the implant. The one or more lobes may extend radially outwardly relative to a conical surface that tapers toward a distal end of the driver engagement portion. In other words, instead of merely forming channels in the conical surface to create an offset lobe having the same circumference as the remainder of the conical surface, the lobe is formed extending outwardly relative to the rest of the conical surface. This configuration can help establish a tighter interconnection between the driver and implant, e.g., to both help prevent the implant from angling relative to the driver during insertion and to efficiently translate driving force from the driver to the implant.

Additionally or alternatively, the driver engagement portion may include a number of lobes less than the number of grooves defined by the implant. The one or more lobes defined by the engagement portion of the driver can be inserted into a subset of the grooves defined by the implant. Grooves of the implant not receiving a corresponding lobe of the driver can be circumscribed by a conical region driver engagement portion (e.g., conical region separating adjacent lobes). Configuring the driver engagement portion with a fewer number of lobes than the number of grooves provided on the implant can be useful for a number of reasons. As one example, the configuration can allow for faster interconnection between the driver and implant within the surgical environment as the clinician can engage the one or more driver lobes with any appropriate set of implant grooves and does not need to align each groove with a corresponding driver lobe. Further, when configured with a taper, different lobes may insect each other toward the distal end of the driver as the cross-sectional size of the driver reduces. By configuring the driver with fewer lobes, interference between adjacent lobes may be reduced or eliminated, allowing the driver to be configured with a tapered profile.

Independent of the specific configuration of the driver and corresponding implant, the driver can be releasably connected to the implant. For example, the driver can be engaged with the implant, the implant driven into a bone using the driver, and the driver then disengaged from the implant to leave the implant in the bone. Detachable driver-implant systems can be used in a variety of different orthopedic cases. In some examples involving small bones, such as bones of the foot for example, a detachable driver-implant system can be used to perform a surgical procedure utilizing a smaller incision (and resulting scar) than if a non-detachable driver-implant system is used during the procedure.

In many procedures, for example, temporary implants (e.g., fixation pins) are inserted into bone to hold the position of one or more bones during subsequent steps of the procedure. The temporary implants are then removed prior to closure of the incision created to access the one or more bones during the procedure. The incision required in these procedures needs to be large enough to provide access to the one or more bones being worked on by the clinician, including accommodating implants, tools, and/or other hardware projecting out of the incision during the surgical procedure.

In accordance with some example techniques of the present disclosure, a surgical technique is provided that utilizes a detachable driver-implant system and comparatively small surgical incision. The clinician can make an incision through the skin of the patient and retract the skin along the incision to expose a first bone region. The clinician can insert an implant into one or more bone portions using a detachable driver-implant system and then detach the driver, leaving the implant in the one or more bone portions. The clinician can reposition the skin of the patient at least partially over the end of the implant to expose a second bone region not exposed while the first bone region was exposed. After optionally performing one or more surgical steps on second bone region, the clinician can reposition the skin to again expose the first bone region (e.g., partially or fully covering the second bone region in the process). The clinician can then reattach the driver to the implant and remove the implant from the one or more bone portions. In this way, a detachable driver-implant system can facilitate a surgical procedure utilizing a smaller incision as compared to a similar procedure in larger incision is made to simultaneously expose both first and second bone regions.

In one example, a tapered lobular surgical driver and implant system is described that includes a bone implant and a driver. The bone implant has a length extending from a proximal end to a distal end, where the proximal end of the implant defines a socket. The socket has a sidewall defining a plurality of grooves and a plurality of alternating protrusions; and the sidewall tapers from the proximal end toward the distal end at an implant angle of taper. The driver includes an engagement portion configured to engage the socket of the bone implant. The engagement portion includes a conical surface tapering at a driver taper angle corresponding to the implant angle of taper and at least one lobe configured to be inserted into one of the plurality of grooves defined by the socket of the bone implant. The example specifies that the lobe projects radially outwardly relative to the conical surface.

In another example, a method of engaging a surgical implant with a tapered lobular surgical driver is described. The method involves inserting an engagement portion of a driver into a socket of a bone implant and rotationally driving the bone implant with the driver. The example specifies that the socket of the bone implant includes a tapered sidewall defining a plurality of grooves and a plurality of alternating protrusions, and the driver engagement portion includes a tapered conical surface and at least one lobe that projects radially outwardly relative to the conical surface.

In another example, a minimally invasive provisional bone fixation technique is described. The technique involves making an incision through a skin of a patient and retracting the skin along the incision to expose at least a portion of a first bone. The technique also includes inserting a bone implant detachably engaged to a driver through the first bone and into a second bone across a joint between the first bone and the second bone. The method includes, after inserting the bone implant, detaching the driver from a proximal end of the bone implant and repositioning the skin of the patient at least partially over the proximal end of the bone implant, thereby exposing a different portion of the joint through the incision. The method further involves attaching a bone fixation device across the different portion of the joint exposed by repositioning of the skin, and after attaching the bone fixation device, performing a second repositioning of the skin to at least partially cover the bone fixation device and re-expose the proximal end of the bone implant through incision. The example further includes engaging the driver with the proximal end of the bone implant and removing the bone implant from the first bone and the second bone.

The details of one or more examples are set forth in the accompanying drawings and the description below. Other features, objects, and advantages will be apparent from the description and drawings, and from the claims.

### BRIEF DESCRIPTION OF DRAWINGS

FIG. 1 is a perspective view of an example surgical driver and implant system that includes a bone implant and a driver.

FIGS. 2 and 3 are perspective and end views, respectively, of the bone implant of FIG. 1 illustrating an example socket configuration.

FIG. 4 is a partial sectional image of the example surgical driver and implant system of FIG. 1 showing the driver partially inserted into the socket of the bone implant.

FIGS. 5A and 5B are perspective and distal end views, respectively, showing an example configuration of an engagement portion of a driver.

FIG. 6 is a sectional view of the driver and bone implant system of FIG. 1 taken along the A-A section line indicated on FIG. 4.

FIG. 7 is a perspective view of the example bone implant of FIG. 1 illustrating an example second driver that can be inserted into a socket of the bone implant.

FIG. 8 is a partial sectional image of the example driver and implant of FIG. 7 showing the driver inserted into the socket of the bone implant.

FIGS. 9A and 9B are perspective and distal end views, respectively, showing another example configuration of an engagement portion of the driver of FIG. 1.

FIGS. 10A and 10B are perspective and distal end views, respectively, showing another example configuration of an engagement portion of the driver of FIG. 1.

FIGS. 11A and 11B are perspective and distal end views, respectively, showing yet another example configuration of an engagement portion of the driver of FIG. 1.

FIG. 12 is a flow diagram illustrating an example surgical technique that can be performed utilizing a detachable driver and implant system.

FIG. 13 is a perspective view of a foot showing example surgical access to one or more bone portions.

FIG. 14 illustrates a first bone region being exposed through an incision while a second bone region where a bone fixation device is desirably attached is covered by the skin of the patient.

FIG. 15 illustrates the skin of a patient being retracted to a different position than shown in FIG. 14 to expose a second bone region while a first bone region is partially or fully covered with the skin.

### DETAILED DESCRIPTION

This disclosure generally relates to orthopedic implants and driver systems, techniques for using such systems, and orthopedic procedures involving detachable implant-driver systems. The orthopedic implants and driver systems can be used in any desired orthopedic procedure involving any one or more bones. In exemplary applications, the systems and/or techniques of the disclosure are may be used during a bone alignment, osteotomy, fusion procedure, fracture repair, and/or other procedures where one or more bones are to be set in a desired position. Such a procedure can be performed, for example, on bones (e.g., adjacent bones separated by a joint or different portions of a single bone) in the foot or hand, where bones are relatively small compared to bones in other parts of the human anatomy. In one example, a procedure utilizing embodiments of the disclosure can be performed during a procedure to correct an alignment between a metatarsal (e.g. a first metatarsal) and a cuneiform (e.g., a medial cuneiform), such as a bunion correction. An example of such a procedure is a lapidus procedure. In another example, the procedure can be performed by modifying an alignment of a metatarsal (e.g. a first metatarsal). An example of such a procedure is a basilar metatarsal osteotomy procedure.

In various examples, the systems and/or techniques of the disclosure may be utilized on comparatively small bones in the foot such as a metatarsal (e.g., first, second, third, fourth, or fifth metatarsal), a cuneiform (e.g., medial, intermediate, lateral), a cuboid, a phalanx (e.g., proximal, intermediate, distal), and/or combinations thereof. For example, a surgical technique may involve inserting an implant through an end of one bone and into an end of an opposing bone separated from each other by a joint. For example, a surgical procedure may involve inserting an implant through a metatarsal and into an end of a cuneiform/cuboid separated from the metatarsal by a tarsometatarsal (“TMT”) joint. As another example, a surgical procedure may involve inserting an implant through an end of one of a metatarsal and a proximal phalanx and into an end of the other bone across a metatarsophalangeal (“MTP”) joint. Other bones and/or bone portions can receive an implant utilizing an implant-driver system according to the disclosure.

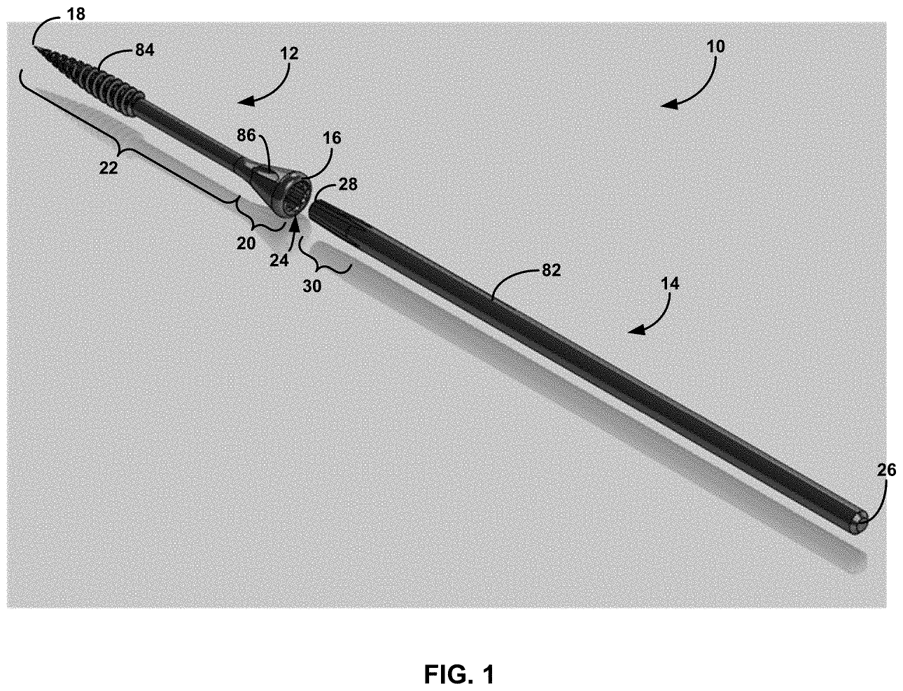

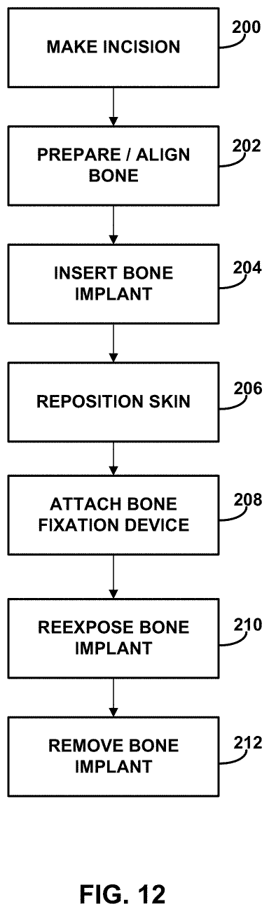

Additional details on example techniques involving insertion of an implant using a detachable driver will be described with respect to FIGS. 12-15. However, an example implant and driver system will first be described with respect to FIGS. 1-11.

FIG. 1 is a perspective view of an example surgical driver and implant system 10 that includes a bone implant 12 and a driver 14. Driver 14 is illustrated as being detached from but insertable into bone implant 12 to form an interconnected assembly that allows rotation of driver 14 to translate through and rotationally drive bone implant 12 into one or more bones. Bone implant 12 has a length extending from a proximal end 16 to a distal end 18, where the distal end provides a leading end making first contact with a bone into which the bone implant is inserted. Bone implant 12 may include a head 20 and a shaft 22. The proximal end 16 of bone implant 12 can define a socket 24 into which driver 14 can be inserted. Driver 14 likewise can define a length extending from a proximal end 26 to a distal end 28. The distal end of the driver can include an engagement portion 30 that is configured to insert into and engage the socket 24 of the bone implant.

As will be described in greater detail, driver and implant system 10 can be configured with a tapered lobular connection in which both socket 24 of bone implant 12 and engagement portion 30 of driver 14 taper from larger cross-sectional area to smaller cross-sectional area moving proximally to distally. Further, engagement portion 30 of driver 14 can define one or more lobes that can be inserted into one or more corresponding grooves of socket 24 of bone implant 12. The one or more lobes defined by engagement portion 30 of driver 14 can be configured to form a robust interconnection with socket 24 of bone implant 12, e.g., minimizing relative movement between the driver and bone implant once engaged together. For example, the interconnection between bone implant 12 and driver 14 may reduce or eliminate angular shifting of the bone implant relative the driver. When angular shifting occurs, distal end 18 of bone implant 12 may shift from being aligned with the axis defined by the length of driver 14 to being angled at a nonzero degree angle relative to the axis. If the of longitudinal axis of bone implant 12 skews relative to the longitudinal axis of driver 14, for example due to a comparatively sloppy drive connection between the bone implant and driver, the bone implant may be driven into one or more bones along a trajectory line that varies from the target directory line intended by the clinicians positioning of driver 14.

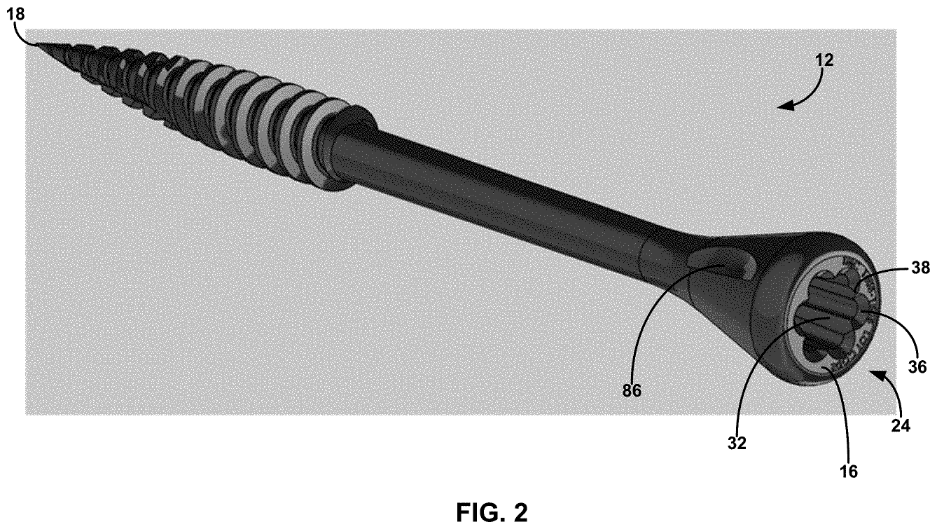

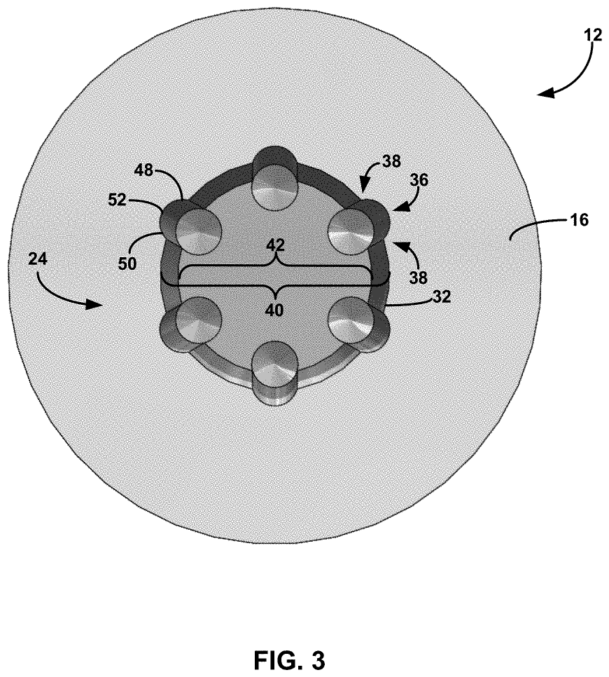

FIGS. 2 and 3 are perspective and end views, respectively, of bone implant 12 illustrating an example configuration of socket 24. Socket 24 can form a bounded cavity defined by a sidewall 32 and a bottom wall 34. Sidewall 32 can define a plurality of grooves 36 and a plurality of protrusions 38, with individual protrusions separating adjacent grooves. Sidewall 32 can extend longitudinally along the length of bone implant 12 from proximal end 16 toward distal end 18. Each groove 36 may be formed by a region of sidewall 32 that extends radially outwardly relative to an adjacent protrusion 38. As a result, the radius of socket 24 from a geometric center to an outermost edge of groove 36 may be larger than the radius of the socket from the geometric center to an outermost edge of protrusion 38.

Sidewall 32 can taper inwardly from proximal end 16 toward distal end 18. For example, socket 24 may define a first diameter 40 at the proximal-most end of the socket and a second diameter 42 at the distal-most end of the socket (e.g., where bottom wall 34 is located). The second diameter 42 can be less than the first diameter 40 as sidewall 32 tapers radially inwardly from the proximal end towards the distal end of the socket. The resulting angle of taper, which may be referred to as an implant angle of taper, may vary depending on the design of the specific socket and bone implant. In some examples, the implant angle of taper ranges from 0.5° to 30° relative to a longitudinal axis bisecting bone implant 12, such as from 1° to 20°, from 2° to 15°, from 3° to 15°, from 2° to 10°, or from 3° to 10°.

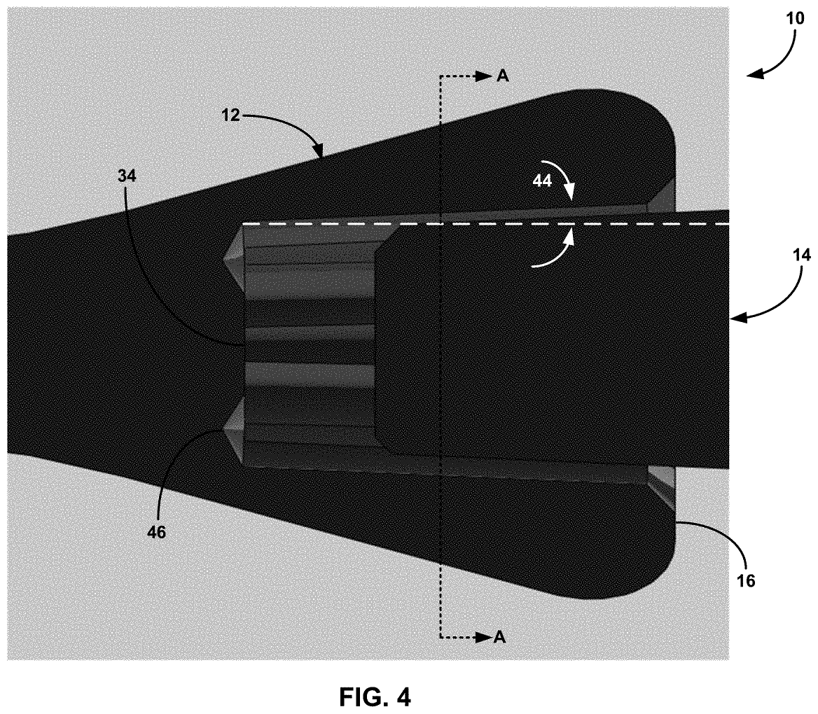

FIG. 4 8 s a partial sectional image of surgical driver and implant system 10 showing driver 14 partially inserted into socket 24 of bone implant 12. FIG. 4 illustrates sidewall 32 tapering from the proximal end 16 of the socket to the distal end of the socket defined by bottom wall 34 to define an implant angle of taper 44. In some implementations, sidewall 32 tapers at a constant angle of taper from the proximal end to the distal end of the socket. In other implementations, sidewall 32 may taper at a variable angle, e.g., such that one lengthwise portion of sidewall 32 tapers at a first angle and another lengthwise portion of sidewall 32 tapers at a second angle different than the first angle.

Bottom wall 34 can define the distal end of socket 24. Bottom wall 34 may be a planar or flat wall, e.g., extending orthogonal to the longitudinal axis of bone implant 12. Alternatively, bottom wall 34 may include one or more sloped or angled surfaces that intersect to define a radius of curvature and/or apex. In the illustrated configuration of FIG. 4, bottom wall 34 is illustrated as a planar wall. Undercuts 46 extend through bottom wall 34 in the region of grooves 36 although, in other examples, such undercuts may not be present.

In different implementations, grooves 36 and/or protrusions 38 may or may not taper at the same implant angle of taper 44 as sidewall 32. As an example, the portion of sidewall 32 between grooves 36 and protrusions 38 may taper at the implant angle of taper 44 while grooves 36 and/or protrusions 38 may not taper or may taper at a different angle than the implant angle of taper. For example, the portion of sidewall 32 between adjacent protrusions 38 may taper at the implant angle of taper 44 while the region of the sidewall defining grooves 36 and protrusions 38 themselves may taper at a second angle of taper different than the implant angle of taper. As another example, the portion of sidewall 32 between adjacent protrusions 38 may taper at the implant angle of taper 44 and the region of the sidewall defining grooves 36 and protrusions 38 may also taper at the implant angle of taper 44. In this configuration, socket 24 can taper about its entire circumferential perimeter at the same angle of taper 44.

With further reference to FIG. 3, in general, each feature described as a groove 36 may be a relatively recessed area (e.g., extending radially outwardly) bounded by a relatively projecting area (e.g., extending radially inwardly) described as a protrusion 38. In some examples, each groove 36 may be defined by a radius of curvature, which may be constant or varying over the perimeter of the groove. In some examples, each grove 36 may be formed by a combination of planar and/or curved surfaces defining the groove.

For example, each groove may include a driving surface 48, a relief surface 50, and a transition surface 52 extending between the driving surface and the relief surface. Driving surface 48 may include a substantially flat face that is configured to be oriented perpendicular to a driving force, e.g., such that the face of the driving surface is substantially parallel to a radial line extending from the center of socket 24. When so configured, driving surface 48 may be configured to receive a driving force applied by a corresponding driving face of driver 14 at a substantially 90° degree angle. This can allow driver 14 to efficiently transfer driving force to bone implant 12 while minimizing slippage between the driver and socket. Other shapes and configurations of socket 24 can be used, however, without departing from the scope of the disclosure.

The specific number and arrangement of grooves 36 and projections 38 protrusions 38 may vary depending on the configuration of bone implant 12. In general, bone implant 12 may include at least two grooves 36 with at least one protrusion 38 between each groove. For example, in different implementations, socket 24 of bone implant 12 may include two grooves, three grooves, four grooves, five grooves, six grooves, seven grooves, or eight or more grooves. Each groove 36 may be separated from each other groove by at least one protrusion 38 (e.g., optionally only one protrusion between each adjacent groove). In some configurations, the multiple grooves 36 are arrayed substantially symmetrically about the perimeter of socket 24, e.g., such that the distance between one groove and an adjacent groove is substantially the same for all grooves in the socket. In other configurations, the multiple grooves may be arrayed asymmetrically about the perimeter of socket 24, e.g., such that the grooves are not position substantially equal distance from each other about the perimeter of socket 24.

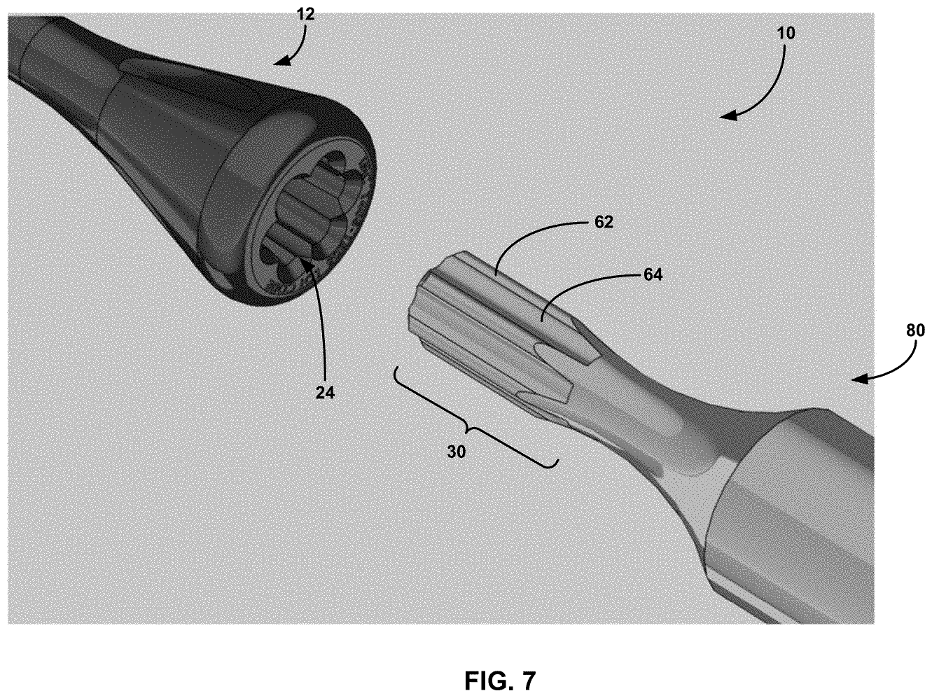

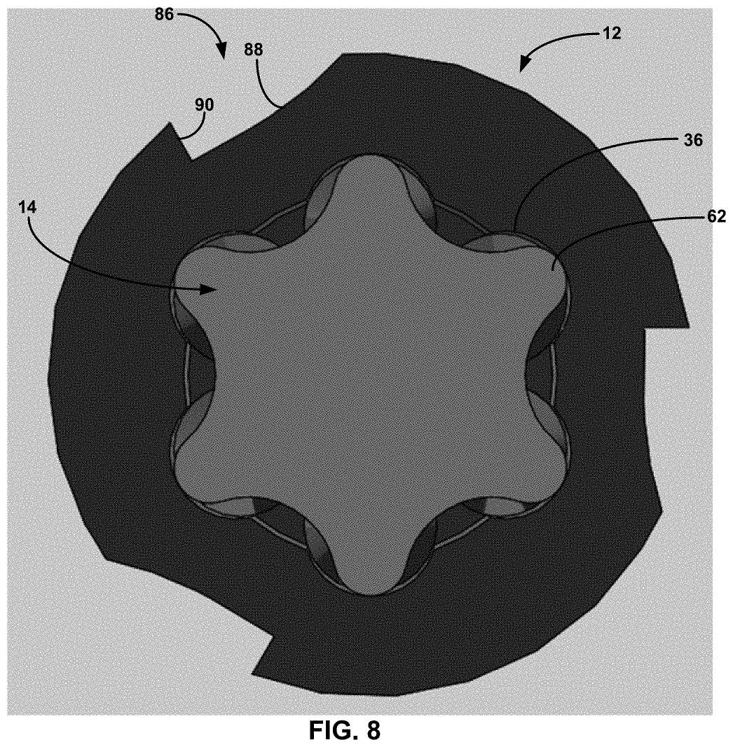

While bone implant 12 can have any desired number of grooves 36, in some implementations, the bone implant has six grooves (e.g., with each groove being separated by a protrusion 38 to provide six protrusions). As we discussed with respect to FIGS. 7 and 8 below, in some examples, socket 24 of bone implant 12 may be designed to engage with multiple different drivers. For example, socket 24 may be configured (e.g., sized and/or shaped) to engage with both driver 14 as well as a standard hexalobular driver. Example hexalobular drivers include those conforming to ISO standard 10664 (1999) and those sold commercially under the tradename Torx®. Configuring bone implant 12 with six grooves may allow socket 24 to receive a hexalobular driver having six lobes, with one lobe of the driver inserted into a corresponding one of each of the six grooves. When so configured, a clinician may use driver 14 for high precision driving of bone implant 12 but may optionally switch to a standard hexalobular driver, e.g., to supplementally drive the bone implant and/or remove the bone implant after high precision placement of the implant is initially achieved using driver 14. This configuration can be effective when using bone implant and driver system 10 as part of a procedure that includes or otherwise utilizes a standard hexalobular driver as part of the instrument kit made available the clinician. For example, the clinician may use a powered driver to which driver 14 is coupled to drive bone implant 12 and switch to a secondary driver having a manually graspable handle (e.g., not attached to a powered driver) to further drive the bone implant.

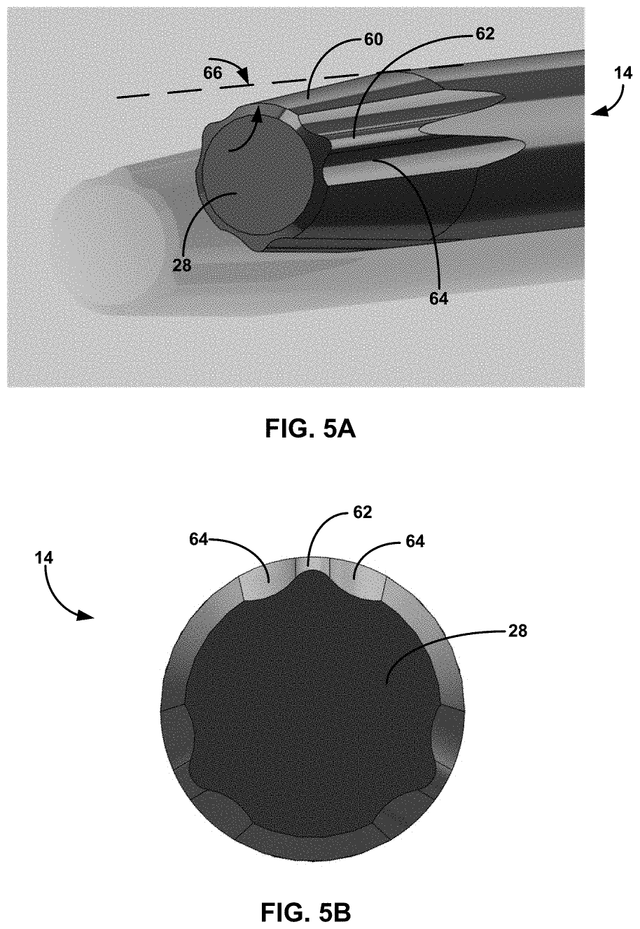

As initially introduced with respect to FIG. 1, bone implant and driver system 10 can include driver 14 that detachably engages with socket 24 of bone implant 12. FIGS. 5A and 5B (collectively referred to as FIG. 5) are perspective and distal end views, respectively, showing an example configuration of engagement portion 30 of driver 14. Engagement portion 30 of driver 14 is insertable into socket 24 of bone implant 12. Engagement portion 30 can taper toward distal end 28 of the driver such that the cross-sectional area of the engagement portion is smaller at the distal end then proximally along the length of the engagement portion.

Engagement portion 30 of driver 14 can define a conical surface 60 and one or more lobes 62 that are offset relative to the conical surface. Conical surface 60 can taper radially inwardly toward a hypothetical convergence point, which is truncated by the distal end 28. Distal end 28 of driver 14 may be a planar or flat wall, e.g., extending orthogonal to the longitudinal axis of driver 14, or the distal end may include one or more sloped or angled surfaces that intersect to define a radius of curvature and/or apex. In either case, conical surface 60 may extend around the entire circumference of engagement portion 30 except where broken by one or more lobes and/or flutes. Each lobe 62 may be configured to be inserted into one of the plurality of grooves 36 forming socket 24 of bone implant 12.

In some configurations, engagement portion 30 includes one or more flutes 64 that are recessed relative to an adjacent lobe 62 and/or conical surface 60. For example, engagement portion 30 can include a flute 64 on each side of a lobe 62, e.g., such that there are at least two flutes between each adjacent lobe. When configured with one or more flutes, the height of a lobe 62 may be measured from the trough of an adjacent flute 64 to an apex of the lobe 62. By contrast, when engagement portion 30 does not include flute 64, the height of a lobe 62 may be measured from conical surface 60 to the apex of the lobe 62.

Conical surface 60 of driver engagement portion 30 can taper from a proximal end of engagement portion 30 toward the distal end 28 of the driver to define a driver taper angle 66 relative to the longitudinal axis of driver 14. In some implementations, conical surface 60 tapers at a constant angle of taper from the proximal end engagement portion 30 to the distal end 28 of the. In other implementations, conical surface 60 may taper at a variable angle, e.g., such that one lengthwise portion of conical surface 60 tapers at a first angle and another lengthwise portion of conical surface tapers at a second angle different than the first angle.

The driver taper angle 66 can correspond to the implant angle of taper 44 defined by the sidewall 32 of bone implant 12. The driver taper angle 66 can correspond to the implant angle of taper 44 by having an angle of taper sufficiently close to that of the implant angle of taper to allow engagement portion 30 of driver 14 to be positioned in socket 24 to a depth at least 50% of the overall depth of the socket, such as at least 60%, at least 70%, or at least 80%. In some implementations, the driver taper angle 66 is the same as the implant angle of taper 44 and may be any value within the example range of values discussed above for implant angle of taper 44. For example, if the implant angle of taper 44 is 10°, the driver taper angle 66 may also be 10°. In other examples, the driver taper angle 66 is different than the implant angle of taper 44 but sufficiently close to still correspond to the angle. For example, driver taper angle 66 may be within a range of ±20% of the implant angle of taper 44, such as ±10%, ±5%, ±3%, or ±2%.

In different implementations, lobes 62 and flute 64 may or may not taper at the same driver taper angle 66 as conical surface 60. As an example, conical surface 60 may taper at the driver taper angle 66 while lobes 62 and/or flutes 64 may not taper or may taper at a different angle than the driver taper angle. For example, lobes 62 and/or flute 64 may taper at an angle of taper different than driver taper angle 66. As another example, lobes 62 and/or flutes 64 may taper at the same driver taper angle 66 as conical surface 60. In this configuration, engagement portion 30 of driver 14 can taper about its entire circumferential perimeter at the same driver taper angle 66.

In some examples, each lobe 62 extends from a geometric center defined by the longitudinal axis of driver 14 a same distance as conical surface 60 or may even be recessed relative to the conical surface. In other configurations, however, each lobe 62 may project radially outwardly relative to conical surface 60. For example, the apex of each lobe 62 may extend farther away from the geometric center defined by the longitudinal axis of driver 14 a greater distance the distance to which conical surface 60 extends (e.g., within a common vertical plane along the length of engagement portion 30).

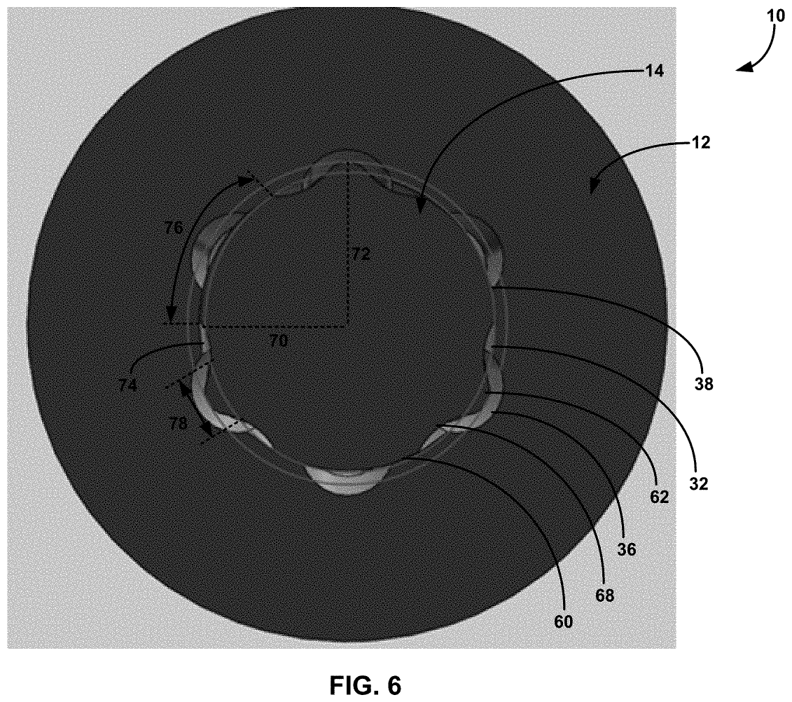

FIG. 6 is a sectional view of driver and bone implant system 10 taken along the A-A section line indicated on FIG. 4. FIG. 6 illustrates an example configuration of the engagement portion of driver 14 inserted into the socket of bone implant 12. In the illustrated configuration, driver 14 includes at least one lobe 62, which is illustrated as being implemented with three lobes, where each a recessed flute is positioned on each side of each lobe. When so configured, each lobe 62 is separated from each other lobe by a first flute, a portion of conical surface 60, and a second flute. In either case, in the illustrated arrangement, the at least one lobe 62 project radially outwardly relative to conical surface 60.

When the engagement portion of driver 14 is inserted into the socket of bone implant 12 according the example configuration of FIG. 6, the one or more lobes 62 are inserted into grooves 36 defined by the bone implant 12. Protrusions 38 defined by the sidewall 32 of bone implant 12 can project radially inwardly toward a convex surface defined by a flute 64 of the driver. Where driver 14 has fewer lobes 62 than the number of grooves 36 defined by bone implant 12, regions of conical surface 60 separating adjacent lobes can circumscribe excess grooves in which a lobe of the driver is not received. This can provide a region of high surface area on driver 14 for engaging the sidewall 32 of the bone implant socket on either side of the groove 36 that is not filled by a lobe of the driver.

In some configurations where driver 14 includes one or more lobes 62 projecting radially outwardly relative to conical surface 60, the conical surface defines a first circumferential perimeter having a first radius 70. The apex (or other most outwardly projecting portion) of the one or more lobes 62 can define a second circumferential perimeter having a second radius 72. The second radius 72 may be greater than the first radius 70 such that the one or more lobes 62 extend radially outwardly relative to the conical surface 60. In some examples, second radius 72 is greater than first radius 70 by at least 0.1 mm, such as at least 0.2 mm, at least 0.5 mm, at least 1 mm, at least 2 mm, or at least 3 mm. It should be appreciated that the foregoing discussion of different radii may be measured and compared within a common cross-sectional plane, since the radius of conical surface 60 and lobe 62 may vary in different cross-sectional planes across the length of driver 14 when configured with a taper.

Each lobe 62 can have a variety of arcuate and/or polygonal shapes. In some implementations, each lobe 62 has an outward surface defining a shape that matches the shape of groove 36 defined by bone implant 12 into which the lobe is to be inserted. For example, each of the plurality of grooves 36 defined by bone implant 12 may be a curved region defined by a radius of curvature. Each lobe 62 defined by the engagement portion of driver 14 may also be a curved region defined by a radius of curvature. The radius of each lobe 62 defined by the engagement portion of driver 14 may be less than or equal to the radius of curvature of each groove 36 defined by the bone implant. In this way, a lobe 62 of driver 14 may be shape indexed to a shape of a receiving groove 36 defined by the bone implant and may also have a size that is equal to or less than the size of the receiving groove 36. This can allow the one or more lobes 62 of driver 14 to be inserted into one or more corresponding grooves 36 of bone implant 12 with sufficient ease.

Engagement portion 30 of driver 14 can have a number of lobes 62 less than or equal to the number of grooves 36 defined by bone implant 12. In one example, driver 14 includes a number of lobes 62 equal to the number of grooves 36 defined by the bone implant such that, when the driver is inserted into the socket of the bone implant, a driver lobe is received in each groove. In other examples, however, driver 14 includes a number of lobes 62 less than the number of grooves 36 defined by the bone implant. When so configured, one or more grooves 36 of bone implant 12 are devoid of a corresponding lobe 62 of driver 14, when the engagement portion of the driver is inserted into the socket of the bone implant.

The specific number of lobes 62 included on driver 14 may vary. In different examples, driver 14 may include a single lobe 62, two lobes 62, three lobes 62, or four more lobes 62. In some implementations where driver 14 includes fewer lobes 62 than the number of grooves 36 defined by bone implant 12, the number of grooves 36 on the bone implant may be at least twice the number of lobes on the driver. For example, if driver 14 includes two lobes 62, bone implant 12 may include at least four grooves 36. As another example, if driver 14 includes three lobes 62, bone implant 12 may include at least six grooves 36. When so configured and engagement portion 30 of driver 14 is inserted into socket 24 of bone implant 12, one half or less of the plurality of grooves 36 are engaged with lobes 62 of the driver and another half or more of the plurality of grooves 36 may be bounded (e.g., circumscribed) by the conical surface 60 of the engagement portion of the driver.

Configuring the engagement portion 30 of driver 14 with a fewer number of lobes 62 than the number of grooves 36 provided on bone implant 12 can be useful for a number of reasons. As one example, the configuration can allow for faster interconnection between the driver and implant within the surgical environment as the clinician can engage the one or more driver lobes with any appropriate set of implant grooves and does not need to align each groove with a corresponding driver lobe. Further, when configured with a taper, different lobes 62 may insect each other toward the distal end 28 of the driver 14 as the cross-sectional size of the driver reduces. In other words, as the cross-sectional size of driver 14 tapers towards distal end 28, the size of conical surface 60 between adjacent lobes 62 may shrink to the point where there is no conical surface separating adjacent lobes and the lobes begin intersecting each other. By configuring driver 14 with a fewer number of lobes 62, the lobes can be spaced farther away from each other to accommodate tapering of the driver.

In some configurations in which driver 14 includes multiple lobes 62, the multiple lobes may be arrayed substantially symmetrically about the perimeter of the driver, e.g., such that each lobe is spaced substantially equal distance from each adjacent lobe. In other configurations, the multiple lobes 62 may be arrayed asymmetrically about the perimeter of the driver, e.g., such that the lobes are not positioned substantially equal distance from each other about the perimeter of driver 14.

The number and spacing of lobes 62 may vary, e.g., based on the size and configuration of driver 14. In some implementations, the one or more lobes 62 defined by driver 14 are sized and spaced such that conical surface 60 defines a greater amount of the circumferential perimeter of the driver than that defined by the one or more lobes. For example, driver engagement portion 30 may define a circumferential perimeter 74 corresponding to the first radius 70. A first portion of the circumferential perimeter 74 may be provided by conical surface 60 positioned along the circumferential perimeter. This can be determined by measuring the amount 76 of each section of conical surface 60 positioned along the circumferential perimeter 74 (one section of which is annotated on FIG. 6) and adding the combined sections together. A second portion of the circumferential perimeter 74 may be provided by the one or more lobes 62 position along the circumferential perimeter. This can be determined by measuring the amount 78 of each lobe position along the circumferential perimeter 74 (one section of which is annotated on FIG. 6) and adding the different lobe amounts together. The first portion defined by conical surface 60 can be greater than the second portion defined by the one or more lobes, which may provide more conical surface 60 in contact with side wall 32 of bone implant 12 than lobe surface. In some examples, the first portion of the circumferential perimeter comprising conical surface 60 is at least 50% of the circumferential perimeter 74, such as at least 60% of the perimeter, or at least 70% of the perimeter.

In use, engagement portion 30 of driver 14 can be inserted into the socket 24 of bone implant 12 (e.g., either by the clinician in a surgical environment or preassembled by a manufacturer). The clinician can then rotationally drive driver 14 to advance bone implant 12 into one or more bones or bones portions. The clinician can then disengage driver 14 from bone implant 12 by removing the engagement portion 30 from the socket of the bone implant.

In some applications, driver and bone implant system 10 is provided with and/or utilized with only a single driver 14. In other applications, however, driver and bone implant system 10 may include a second driver having a different configuration than driver 14. For example, introduced above, socket 24 may be configured (e.g., sized and/or shaped) to engage with both driver 14 as well as a hexalobular driver. Example hexalobular drivers include those conforming to ISO standard 10664 (1999) and those sold commercially under the tradename Torx®. Configuring bone implant 12 with six grooves may allow socket 24 to receive a hexalobular driver having six lobes, with one lobe of the driver inserted into a corresponding one of each of the six grooves. Configuring socket 24 of bone implant 12 to mate with a second driver can provide flexibility and adaptability for the clinician to utilize different instruments in the surgical environment (e.g., depending on preference and/or availability) when inserting and/or removing bone implant 12 from one or more bones. In some such configurations, a clinician may use driver 14 for high precision driving of bone implant 12 but may optionally switch to a different hexalobular driver, e.g., to supplementally drive the bone implant and/or remove the bone implant after high precision placement of the implant is initially achieved using driver 14. For example, the clinician may use a powered driver to which driver 14 is coupled to drive bone implant 12 and switch to a secondary driver having a manually graspable handle (e.g., not attached to a powered driver) to further drive the bone implant.

FIG. 7 is a perspective view of bone implant 12 illustrating an example second driver 80 that can be inserted into socket 24 of the bone implant in lieu of driver 14 to drive the implant. FIG. 8 is a partial sectional image of the example driver and implant of FIG. 7 showing driver 80 inserted into the socket of the bone implant. As shown in FIGS. 7 and 8, a second driver 80 can be provided with bone implant 12 (e.g., in addition to driver 14), where the second driver has a number of lobes 62 equal to the number of grooves 36 defined by the socket 24 of the bone implant. For example, where socket 24 of bone implant 12 is a hexalobular socket having six grooves 36 (e.g., optionally with six protrusions 38), driver 80 may be a hexalobular driver having six lobes 62 (e.g., optionally with six flutes 64). Driver 80 may define a straight sidewall, e.g., such that the engagement portion 30 of the driver does not taper from a proximal end to a distal end but rather has a substantially constant cross-sectional size (e.g., area) over the length of engagement portion.

Socket 24 of bone implant 12 can receive the engagement portion 30 of driver 14 having a tapered cross-sectional size that is comparatively larger in a proximal region and comparatively smaller in a distal region, or can receive the engagement portion 30 of driver 80 having a constant cross-sectional size over the portion of the length of the driver insertable into the socket. In some such systems, driver 14 may have a number of lobes 62 less than number of grooves 36 defined by the socket of the bone implant, whereas driver 80 may have a number of lobes 62 equal to the number of grooves 36 defined by the socket of the bone implant. In some examples, driver 80 may be a hexalobular driver having a standard size of either a T-6, T-7, T-8, T-9, T-10, or larger driver. Socket 24 of bone implant 12 may be sized to receive and engage the hexalobular driver of any one of these corresponding standard sizes.

With further reference to FIG. 1, driver 14 is illustrated as including a shaft 82 that defines a longitudinal axis extending from the proximal end 26 to the distal end 28. In some implementations, engagement portion 30 extends the entire length of the driver shaft. In other implementations, engagement portion 30 extends over a portion of the driver shaft that is less than the entire length, such as 50% or less of the shaft length, 40% or less of the shaft length, 30% or less of the shaft length, 20% or less of the shaft length, or 10% or less of the shaft length. The one or more lobes 62 and/or flutes 64 (FIG. 5) on the engagement portion 30 of the driver can extend all the way to the distal end 28 of the driver shaft 82 (e.g., over engagement portion) or may extend partially along the length of the shaft but terminate prior to the distal end 28 of the shaft.

Driver 14 can be physically grasp by a clinician and rotated under hand power to drive bone implant 12 to which the driver is attached. In these configurations, driver 14 may include a handle (e.g., a cylindrical handle having ribbing or a textured surface for grasping, a T-bar) about proximal end 26 of the driver. Additionally or alternatively, driver 14 may include a connection about proximal end 26 configured to operatively connect the shaft 82 to a powered driver. The powered driver can be a handheld unit with trigger and motor that is powered by a power source (e.g., electricity, pneumatic, hydraulic) to rotate the shaft of the driver.

While driver 14 can utilize any types of connection interfaces, in some implementations, the driver may include an AO quick connect interface having a D-shaped profile. A flat portion defined by the D-shaped profile can engage another flat portion in an inner cavity of the AO quick connect interface on the powered driver. This engagement prevents rotation of the shaft with respect to the interface. The shaft may be retained in the interface by the operation of ball bearings. Two ball bearings can be positioned radially about a central axis of the interface, about 180 degrees apart. The ball bearings may engage a groove formed in the end of the shaft.

Bone implant 12 can have any configuration that allows the implant to be inserted into a bone. Bone implant 12 can be in the form of a pin (e.g., having a substantially constant cross-sectional size across the length of the pin), a screw, and/or other mechanical fixation device that can be inserted into a bone (e.g., via rotational driving). In the illustrated example of FIG. 1, bone implant 12 is shown as a screw having head 20 in shaft 22. Head 20 may be a region of enlarged cross-sectional area (e.g., in a widthwise direction orthogonal to the longitudinal axis of the implant relative to the shaft).

Shaft 22 of bone implant 12 (e.g., when configured with or without head 20) may include threading 84 partially or fully encircling the shaft for engaging with a bone portion into which the implant is to be inserted. Threading 84 may be a helical structure used to convert rotational motion into linear movement or force. Threading 84 may be defined by a ridge of material wrapped around an inner cylinder or cone of material in the form of a helix, to define a straight thread or a tapered thread. In some examples, threading 84 is self-tapping and/or shaft 22 may taper at the distal end 18 to facilitate ease of insertion of the bone implant into a bone. In various examples, bone implant 12 may be configured as a single-lead screw or as a multi-lead screw, such as a dual lead screw, a tri-lead screw, or a quad-lead screw, such that each time the body of the screw is rotated one turn, it is advanced axially by a multiple of the pitch or spacing between adjacent ridges.

When bone implant 12 includes threading 84, the bone implant may be threaded along the entire length of shaft 22 or may be threaded along less than the entire length of the shaft. For example, bone implant 12 may be threaded along less than three quarters of the length of the shaft, less than half of the length of the shaft, or less than a quarter of the length of the shaft.

When bone implant 12 is configured with an enlarged head 20, the head may also be threaded to provide a locking screw that can be engaged with counter threading of a bone plate or other screw receiving opening (e.g., locking bone plate). Alternatively, head 20 may be unthreaded, e.g., to provide a compression screw. In some examples, bone implant 12 may include one or more cut outs 86 (e.g., FIGS. 1 and 8) adjacent the proximal end 16 of the implant, such as on head 20 of the implant. The one or more cut outs 86 may include a ramped transition surface 88 and a cutting surface 90, e.g., extending substantially perpendicularly relative to the face of the bone to be cut during insertion of the bone implant. As the enlarged head 20 of bone implant 12 begins advancing into a bone, the one or more cut outs 86 can help cut an opening in the bone for insertion of the bone implant.

Bone implant 12 can having a variety of different sizes depending on the desired application. In some applications, bone implant 12 has a length (from the proximal end 16 to the distal end 18) ranging from 8 mm to 18 mm, such as from 10 mm to 16 mm, or from 12 mm to 14 mm. Although the diameter of the bone implant may vary, in some examples, the diameter of shaft 22 ranges from 2 mm to 4 mm, such as from 2.5 mm to 3.2 mm.

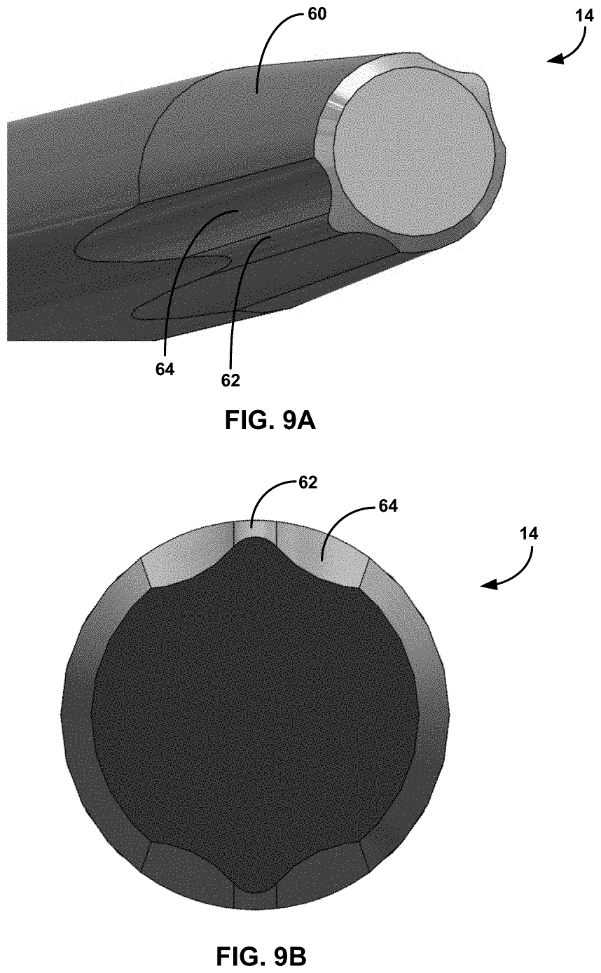

Engagement portion 30 of driver 14 can have a number of different lobe and/or flute configurations, as discussed above. FIGS. 9A and 9B are perspective and distal end views, respectively, showing another example configuration of the engagement portion of driver 14. As shown in this example, the engagement portion of driver 14 includes two lobes 62 spaced on opposite sides (e.g., 180° apart) about the perimeter of the driver. A pair of flutes 64 bounds each lobe, with a region of conical surface 60 extending between flutes.

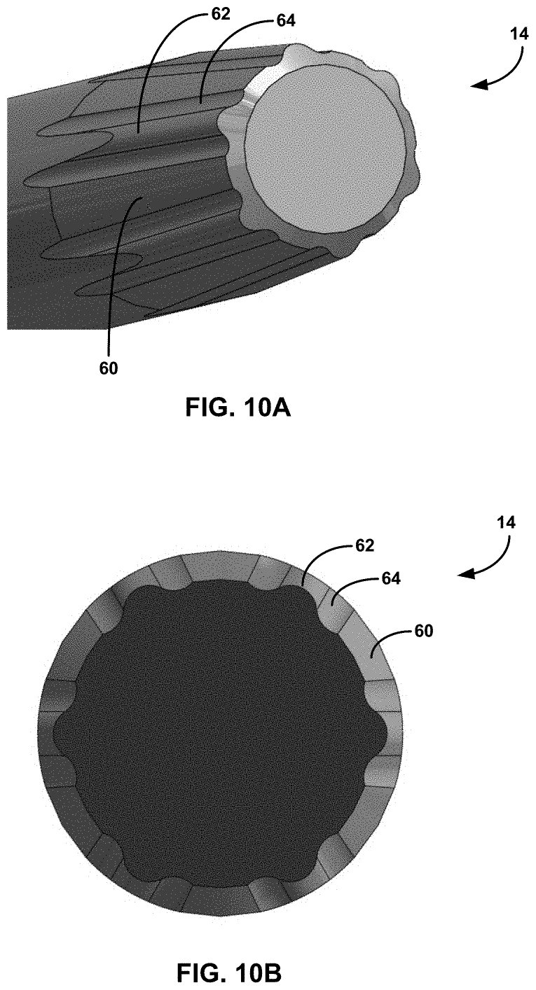

FIGS. 10A and 10B are perspective and distal end views, respectively, showing yet another example configuration of the engagement portion of driver 14. As shown in this example, the engagement portion of driver 14 includes six lobes 62 spaced substantially equal distance from each other about the perimeter of the driver. A pair of flutes 64 bounds each lobe, with a region of conical surface 60 extending between flutes.

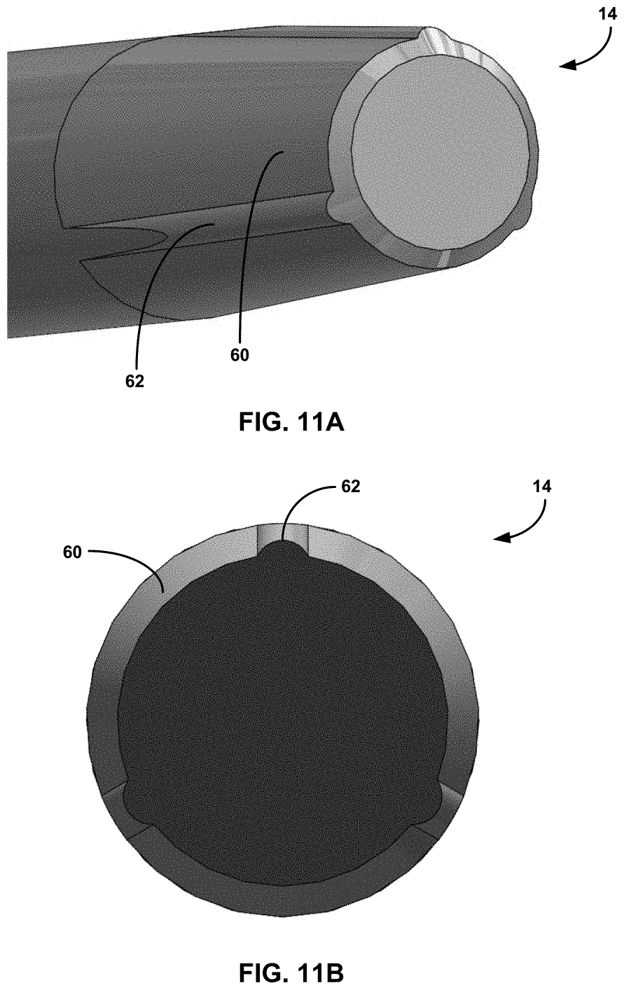

FIGS. 11A and 11B are perspective and distal end views, respectively, showing yet another example configuration of the engagement portion of driver 14. As shown in this example, the engagement portion of driver 14 includes three lobes 62 spaced substantially equal distance from each other about the perimeter of the driver. In this illustrated example, each lobe 62 is separated from each other lobe by a region of conical surface 60. However, driver 14 in this example does not include flutes 64.

Bone implant 12 and driver 14 can have a variety of configurations as described herein other than those specifically illustrated. For example, while socket 24 of bone implant 12 and engagement portion 30 of driver 14 are generally illustrated as having a circular cross-sectional shape (e.g., with lobes, flutes, grooves, and/or protrusions interrupting the circular perimeter), one or both can may have a variety of other arcuate (e.g., square, rectangular, hexagonal) or curved (e.g., oval) cross-sectional shapes and/or combinations of arcuate and curve cross-sectional shapes without departing from the scope of the disclosure.

In use, driver and implant system 10 may come preassembled with the engagement portion of the driver inserted into the socket of the bone implant (e.g., within a sterile kit). Alternatively, bone implant 12 and driver 14 may be provided as separate devices that the clinician can engage together to form a combined structure for driving the bone implant into one or more bones are bone portions using the driver.

To attach driver 14 to bone implant 12, the engagement portion 30 of the driver can be advanced axially into the socket 24 of the bone implant. The engagement portion 30 of the driver can be pressed into the socket into a tight fit is established between the driver and the socket (e.g., such that the driver cannot be advanced farther into the socket under hand pressure). Depending on the configuration of engagement portion 30 of driver 14 and socket 24 of bone implant 12, a tapered engagement portion may fit into a corresponding tapered socket. The tapered engagement portion 30 may be configured (e.g., size and/or shaped) relative to the tapered socket 24 such that each lobe of the engagement portion contacts the sidewall 32 defining a corresponding groove 36 over the entire length of the lobe inserted into the socket.

In some configurations, engagement portion 30 of driver 14 includes fewer lobes than the number of grooves defined by the socket 24 of bone implant 12. Accordingly, insertion of driver 14 into bone implant 12 may cause some but not all of the grooves 36 defined by the bone implant to receive lobes of the driver. The remaining grooves 36 may be circumscribed (e.g., bounded, closed, crossed over) by regions of conical surface 60 extending between adjacent lobes 62 and/or flutes 64 of the driver.

Detachable driver and implant systems, including those configured as described herein with respect to driver and implant system 10, can be used in a variety of different orthopedic procedures. For example, a detachable driver and implant system may be used to temporarily or permanently install one or more bone implants into and/or through one or more bones or bones portions during a surgical procedure. After suitably inserting the implant into one or more bones or bones portions, the clinician can detach the driver from the implant to leave the implant in the one or more bones are bone portions. If the bone implant is a temporary implant, for example installed for purposes of holding one or more bones or bones portions during subsequent portions of the surgical procedure, the clinician may subsequently reengage the bone implant with the same or different driver used originally install a bone implant. The clinician can then remove the bone implant (e.g., drive the bone implant in a reverse rotational direction from the rotational direction used to install the bone implant) from the one or more bones or bones portions into which the bone implant was installed.

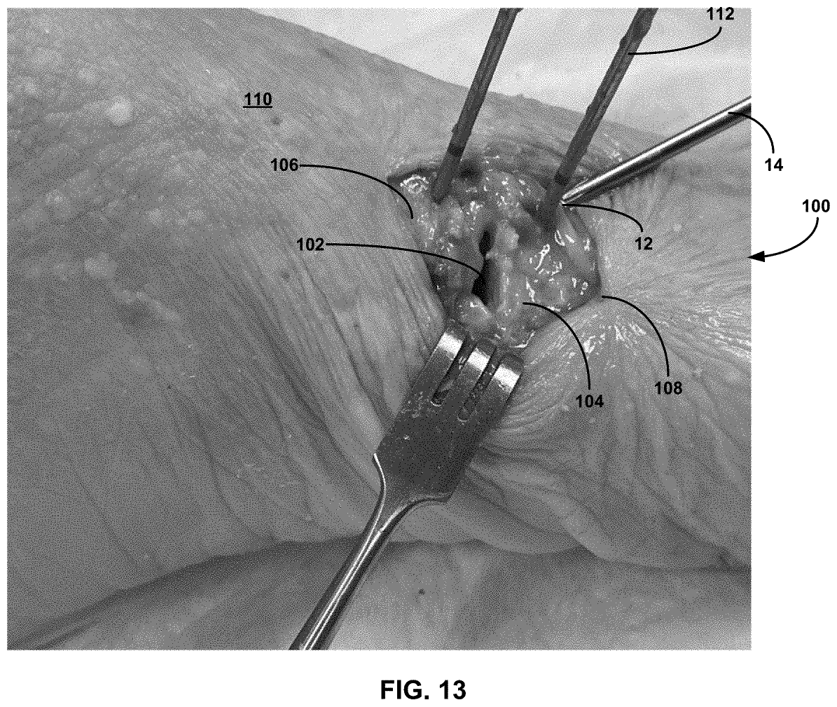

FIG. 12 is a flow diagram illustrating an example surgical technique that can be performed utilizing a detachable driver and implant system. The example technique of FIG. 12 will be described with respect to example procedure steps illustrated in FIGS. 13-15. For purposes of discussion, the example technique of FIG. 12 will be described with respect to driver and implant system 10 utilizing driver 14 and bone implant 12. It should be appreciated, however, that the technique of FIG. 12 may be performed with any driver and implant configuration where the driver can be releasably connected to the implant to insert and/or remove the implant from bone, and the technique is not limited to the specific driver and implant system described herein.

For example, the technique of FIG. 12 may be performed using any driver and bone implant where one of the driver and the implant has a male connection feature and the other of the driver in the implant has a complementary female connection feature configured to detachably coupled to the male connection feature. Example connection features may include standard hexalobular or star-shaped male and female complementary connectors; square-shaped or other polygonal shaped male and female complement three connectors; or yet other types of connection configurations whereby a driver can detachably connect to a bone implant.

The example technique of FIG. 12 may be performed to provide access to multiple different regions of one or more bones or bones portions through a comparatively small incision using a detachable driver and a bone implant system. The clinician may access two or more different regions of the bones and/or bone portions through the incision by manipulating retraction of the skin on either side of the incision. For example, the clinician may preferentially pull or retract the skin on one side of the incision to provide access to a first region, release the pulling force of the skin on that side of the incision, and then preferentially pull or retract the skin on the other side of the incision to provide access to a second region. The two regions of the bones and/or bone portions may not be simultaneously accessible at the same time through the single incision. While a larger incision may allow simultaneous access to both of the two or more regions, this would necessitate a longer incision and resulting scar line for the patient.

The example technique of FIG. 12 will be described with respect to access to the tarsometatarsal joint separating a metatarsal from an opposed bone (e.g., cuneiform), particularly the first metatarsal from the medial cuneiform. The technique can be performed on other bones or bones portions in the foot, such as a metatarsophalangeal joint separating a metatarsal from opposed phalanx, or two portions of the same bone (e.g., two different portions of a metatarsal or other bone, such as when the bone as been cut into two or more different portions via an osteotomy or broken into two or more portions via fracture). However, the technique of FIG. 12 is not limited to such example anatomical locations and may be performed on any two bone portions separated from each other by a joint and/or two or more bone portions.

FIG. 13 is a perspective view of a foot 100 showing example surgical access to one or more bone portions, which is illustrated as surgical access to a TMT joint 102 separating a metatarsal 104 from an opposed cuneiform 106. With reference to FIGS. 12 and 13, the example technique involves surgically accessing one or more bones (e.g., a single bone that may subsequently be cut to form a joint between two portions of the bone; two different bones separated by a native joint), which is illustrated as accessing a TMT joint 102 separating metatarsal 104 from cuneiform 106. To surgically access the joint, the patient may be placed in a supine position on the operating room table and general anesthesia or monitored anesthesia care administered. Hemostasis can be obtained by applying thigh tourniquet or mid-calf tourniquet. An incision 108 can be made through the skin 110, such as on a dorsal side of the foot, a medial side of the foot, or on a dorsal-medial side of the foot (200).

While the specific length of incision 108 (e.g., in the distal to proximal direction parallel to the long axis of metatarsal 104) may vary depending on the anatomy of the specific patient undergoing the procedure and the clinician performing the surgery, in some examples, the incision may have a length less than 20 cm, such as less than 15 cm, or less than 12 cm. For example, the clinician may cut incision 108 to have a length less than 10.5 cm, less than 8 cm, less than 6 cm, or less than 5 cm. In some instances, incision 108 may have a length ranging from 1 cm to 12 cm, such as from 1.5 cm to 8 cm, 2 cm to 6 centimeters, or from 3 cm to 4 cm.

With one or more bones are bone portions exposed through incision 108, the technique of FIG. 12 involves optionally preparing and/or realigning one or more bones (202). For example, the clinician may the end of each bone forming a joint so as to promote fusion of the bone ends across the joint following surgery (e.g., realignment). Bone preparation may involve using a tissue removing instrument to apply a force to the end face of the bone so as to create a bleeding bone face to promote subsequent fusion. Example tissue removing instruments that can be used include, but are not limited to, a saw, a rotary bur, a rongeur, a reamer, an osteotome, a curette, and the like. The tissue removing instrument can be applied to the end face of the bone being prepared to remove cartilage and/or bone. For example, the tissue removing instrument may be applied to the end face to remove cartilage (e.g., all cartilage) down to subchondral bone. Additionally or alternatively, the tissue removing instrument may be applied to cut, fenestrate, morselize, and/or otherwise reshape the end face of the bone and/or form a bleeding bone face to promote fusion. In instances where a cutting operation is performed to remove an end portion of a bone, the cutting may be performed freehand or with the aid of a cutting guide having a guide surface positionable over the portion of bone to be cut. When using a bone preparation guide, a cutting instrument can be inserted against the guide surface (e.g., between a slot define between two guide surfaces) to guide the cutting instrument for bone removal.

In some examples, the clinician cuts at least one bone defining the TMT joint 102 (e.g., one or both of metatarsal 104 and cuneiform 106). The clinician may cut both bones defining the joint or may cut only one bone defining the joint and perform a different preparation technique on the other bone. The clinician can prepare the end face of one or both bones and/or realign one bone relative to another bone in any order. For example, the clinician can prepare the end face of either the metatarsal or the cuneiform before preparing the end face of the other bone. Further, one or both of the end faces of the metatarsal and the cuneiform can be prepared before and/or after the metatarsal is moved relative to the cuneiform.

In some examples, a clinician may insert the two pins 112 (e.g., parallel pins): one on each side of joint 102. The two pins may be used to position hardware over one or both bones, such as a bone preparation guide and/or compressor. Additionally or alternatively, one or both pins may be used to manipulate a position of one or both bones. Details on example bone realignment instruments and techniques that can be used in conjunction with the present disclosure are described in U.S. Pat. No. 9,622,805, issued Apr. 18, 2017 and entitled “BONE POSITIONING AND PREPARING GUIDE SYSTEMS AND METHODS” the entire contents of which are incorporated herein by reference. Details on example compressing instruments that may be used can be found in US Patent Publication No. 2020/0015856, published Jan. 16, 2020 and entitled “COMPRESSOR-DISTRACTOR FOR ANGULARLY REALIGNING BONE PORTIONS,” the entire contents of which are incorporated herein by reference.

Before or after preparing one or both ends of metatarsal 104 and cuneiform 106, the clinician may move the metatarsal in at least one plane. For example, the clinician may move metatarsal 104 in at least the transverse plane to close an intermetatarsal angle between the metatarsal and an adjacent bone (e.g., a second metatarsal) and/or a frontal plane (e.g., to reposition the sesamoid bones substantially centered under the metatarsal). In some examples, the clinician moves the bone portion in multiple planes, such as the transverse plane and/or frontal plane and/or sagittal plane. The clinician may or may not utilize a bone positioning device to facilitate movement of the bone portion. In some examples, the clinician manually moves metatarsal 104 in addition to or moving the metatarsal through a force applied by a bone positioner.

In general, an anatomically aligned position means that an angle of a long axis of a first metatarsal relative to a long axis of a second metatarsal is about 10 degrees or less in the transverse plane or sagittal plane. In certain embodiments, anatomical misalignment can be corrected in both the transverse plane and the frontal plane. In the transverse plane, a normal intermetatarsal angle (“IMA”) between a first metatarsal and a second metatarsal is less than about 9 degrees. An IMA of between about 9 degrees and about 13 degrees is considered a mild misalignment of the first metatarsal and the second metatarsal. An IMA of greater than about 16 degrees is considered a severe misalignment of the first metatarsal and the second metatarsal. In some embodiments, metatarsal 104 is moved to reduce the IMA from over 10 degrees to about 10 degrees or less (e.g., to an IMA of about 1-5 degrees), including to negative angles of about −5 degrees or until interference with the second metatarsal, by positioning the first metatarsal at a different angle with respect to the second metatarsal.

With respect to the frontal plane, a normal metatarsal will be positioned such that its crista prominence is generally perpendicular to the ground and/or its sesamoid bones are generally parallel to the ground and positioned under the metatarsal. This position can be defined as a metatarsal rotation of 0 degrees. In a misaligned first metatarsal, the metatarsal is axially rotated between about 4 degrees to about 30 degrees or more. In some embodiments, the metatarsal is repositioned in the frontal plane by reducing the metatarsal rotation from about 4 degrees or more to less than 4 degrees (e.g., to about 0 to 2 degrees) by rotating the metatarsal with respect to the medial cuneiform.

In some applications, independent of whether the clinician performs a specific bone realignment technique discussed above, the clinician may compress the end faces of the prepared bones (e.g., metatarsal 104, cuneiform 106) together across the joint 102 between the bones. For instance, the clinician may attach a compressing instrument to metatarsal 104 with one or more fixation pins and also attach the compressing instrument to cuneiform 106 using one or more fixation pins. In some examples, the clinician lifts a bone preparation guide off two or more pins that extend parallel to each other, at least one of which is inserted into the metatarsal and at least one of which is inserted into the cuneiform and then installs a compressor back down on the parallel pins.

Independent of whether the clinician optionally prepares and/or realigns one or more bones or bones portions, the technique of FIG. 12 may involve inserting a bone implant detachable engaged to a driver through one bone and into a second bone, e.g., to temporarily or provisionally fixate a position of the bones relative to each other (204). For example, the clinician may insert a bone implant through an end face of one bone (e.g., metatarsal 104) and into an adjacent bone (e.g., an adjacent metatarsal, an end face of an opposed cuneiform 106), with the bone implant crossing joint 102 between the two bones.

For example, with reference to FIG. 13, the clinician may retract skin 110 along incision 108 to expose a first region of one or more bones and insert bone implant 12 through one bone and into an adjacent bone across joint 102 through the first expose region of the bones. The clinician can engage driver 14 to drive bone implant 12 into the first bone (e.g., metatarsal 104) and into a second bone (e.g., cuneiform 106). In some examples, the clinician drives bone implant 12 into one or both bones until the proximal end of the bone implant is substantially flush with (e.g., flush with) or recessed relative to the surface of the bone. Clinician can install bone implant 12 such that a portion of the bone implant is positioned in the first bone, a portion of the bone implant is positioned in the second bone, and the bone implant crosses the joint between the two bones. In this way, the bone implant 12 can hold the position of the first bone relative to the second bone across the joint.

After installing bone implant 12 to a suitable depth into the first bone and the second bone, the clinician can detach driver 14 from the bone implant, leaving the bone implant in the bones. In the technique of FIG. 12, the clinician may then reposition the skin 110 to partially or fully cover the distal end of the bone implant (206) and, optionally in the process, expose a second region of one or both bones not initially exposed when the first region was exposed (206).

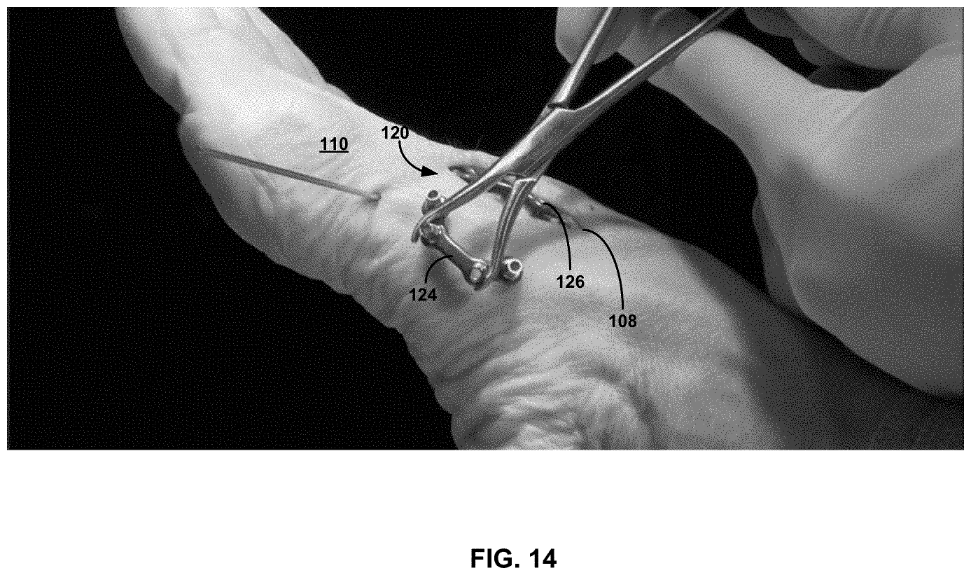

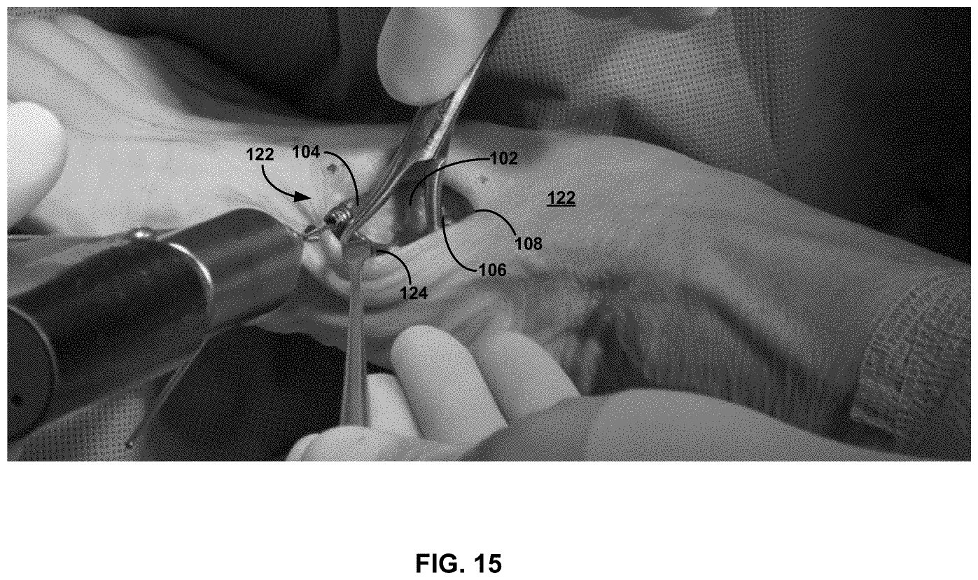

FIGS. 14 and 15 illustrate two different skin retraction positions that can be performed for exposing different regions of one or more bones skin 110. FIG. 14 illustrates a first bone region 120 (e.g., which is illustrated as being on a dorsal side of a foot) being exposed through incision 108 while a second bone region where a bone fixation device 122 is desirably attached is covered by the skin 110. FIG. 15 illustrates skin 110 being retracted to a different position exposing a second bone region 124 through incision 108 (e.g., which is illustrated as being on a medial side of the foot) while the first bone region 120 is partially or fully covered with skin 110. The size of incision 108 may be sufficiently small that skin 110 cannot be retracted sufficiently far apart to simultaneously expose both first bone region 120 and second bone region 122.

With the second bone region 122 exposed, the clinician may attach a bone fixation device 124 across joint 102 over region of the joint other than that exposed by first bone region 120. Bone fixation device 124 can be attached to the first bone (e.g., metatarsal 104) in the second bone (e.g., cuneiform 106) to permanently fixate the positions of the bone relative to each other and/or to promote subsequent fusion between the bones. Example bone fixation devices include, but are not limited to, a bone screw (e.g., a compressing bone screw), a bone plate, a bone staple, a pin (e.g., an intramedullary implant), and/or combinations thereof.

After attaching bone fixation device 124, the clinician can reposition skin 110 to at least partially cover second bone region 122 and/or bone fixation device 124 (e.g., fully covering second bone region 12s and/or bone fixation device 124), re-exposing first bone region 120 and/or the proximal end of bone implant 12 (210). In some examples, the clinician may attach an additional bone fixation device 126 to the first and second bones across the joint 102 in the first bone region 120 before or after attaching bone fixation device 124 and the second bone region 122. In either case, the clinician can engage driver 14 (or a different driver, such as secondary driver 80) with the bone implant 12 through incision 108 with first bone region 120 exposed. The clinician can then use the driver to remove bone implant 12 from first bone 104 and second bone 106 (212). After performing any optional further procedural steps, the clinician can close incision 108. In this way, a detachable driver and implant system can facilitate a minimally invasive surgical procedure, limiting the length of incision on a patient and corresponding scar line.

Various examples have been described. These and other examples are within the scope of the following claims.

1. A tapered lobular surgical driver and implant system, the system comprising:
(a) a bone implant having a length extending from a proximal end to a distal end, the proximal end of the implant defining a socket wherein:
(i) the socket has a sidewall defining a plurality of grooves and a plurality of alternating protrusions; and
(ii) the sidewall tapers from the proximal end toward the distal end at an implant angle of taper; and
(b) a driver comprising an engagement portion configured to engage the socket of the bone implant, wherein:
(i) the engagement portion comprises a conical surface tapering at a driver taper angle corresponding to the implant angle of taper; and
(ii) the engagement portion comprises at least one lobe configured to be inserted into one of the plurality of grooves defined by the socket of the bone implant, the at least one lobe projecting radially outwardly relative to the conical surface.

2. The system of claim 1, wherein the engagement portion has a number of lobes less than a number of the plurality of grooves.

3. The system of claim 1, wherein the driver engagement portion defines a circumferential perimeter, the conical surface comprises a first portion of the circumferential perimeter, and the at least one lobe comprises a second portion of the circumferential perimeter, the first portion being greater than the second portion.

4. The system of claim 3, wherein the first portion of the circumferential perimeter comprising the conical surface is at least 50% of the circumferential perimeter.

5. The system of claim 1, wherein the driver further comprises a flute on each side of each lobe on the engagement portion, the flute being recessed relative to the conical surface.

6. The system of claim 1, wherein the engagement portion of the driver comprises at least two lobes spaced equidistant about a perimeter of the engagement portion.

7. The system of claim 6, wherein each of the at least two lobes are spaced from each other lobe by a portion of the conical surface.

8. The system of claim 1, wherein the at least one lobe projects radially outwardly relative to the conical surface by:
the conical surface defining a first circumferential perimeter having a first radius;
an apex of each of the at least one lobe defining a second circumferential perimeter having a second radius; and
the second radius is greater than the first radius.

9. The system of claim 1, wherein the engagement portion has a number of lobes less than a number of the plurality of grooves, and the number of the plurality of grooves on the implant is at least twice the number of lobes on the driver such that, when the engagement portion is inserted into the socket of the implant, one half or less of the plurality of grooves are engaged with lobes from the driver and another half or more of the plurality of grooves are bounded by the conical surface of the engagement portion.

10. The system of claim 1, wherein the socket is a hexalobular socket having six grooves and six protrusions.

11. The system of claim 10, wherein the engagement portion of the driver has three lobes spaced substantially equidistant about a perimeter of the engagement portion.

12. The system of claim 10, wherein the driver comprises a first driver, and further comprising a second hexalobular driver comprising an engagement portion defining a straight sidewall and six lobes, the six lobes being insertable into corresponding ones of the six grooves of the bone implant socket.

13. The system of claim 1, wherein each of the plurality of grooves of the bone implant has a radius of curvature, and each lobe of the engagement portion of the driver has a radius of curvature less than or equal to the radius of curvature of the plurality of grooves.

14. The system of claim 1, wherein the plurality of grooves and the plurality of alternating protrusions each taper at the implant angle of taper.

15. The system of claim 1, wherein each lobe of the engagement portion tapers at the driver taper angle.

16. The system of claim 1, wherein the driver angle taper and the implant angle of taper are a same angle.

17. The system of claim 1, wherein the driver angle taper and the implant angle of taper are each in a range from 0.5 degrees to 30 degrees.

18. The system of claim 1, wherein the socket comprises a planar bottom wall and a distal end of the engagement portion comprises a planar wall.

19. The system of claim 1, wherein the driver comprises a shaft defining a longitudinal axis extending from a distal end comprising the engagement portion to a proximal end, and the proximal end comprises a handle configured to be manually gripped by a user to apply rotational motion to the shaft.

20. The system of claim 1, wherein the driver comprises a shaft defining a longitudinal axis extending from a distal end comprising the engagement portion to a proximal end, and the proximal end comprises a connector configured to operatively connect the shaft to a powered driver.

21. The system of claim 1, wherein the distal end of the implant comprises threading.

22. The system of claim 21, wherein the bone implant comprises a bone screw having a head and a shaft, the head having a larger cross-sectional area than the shaft, wherein the head comprises the proximal end of the implant defining the socket and the shaft comprises the distal end of the implant comprising threading.

23. The system of claim 22, wherein the shaft of the bone implant is threaded along less than half of a length of the shaft.

24. A method of engaging a surgical implant with a tapered lobular surgical driver, the method comprising:
inserting an engagement portion of a driver into a socket of a bone implant, the socket of the bone implant comprising a tapered sidewall defining a plurality of grooves and a plurality of alternating protrusions, the driver engagement portion comprising a tapered conical surface and at least one lobe that projects radially outwardly relative to the conical surface; and
rotationally driving the bone implant with the driver.

25. The method of claim 24, wherein inserting the engagement portion of the driver into the socket of the bone implant comprises engaging at least one and less than all of the plurality of grooves of the implant socket with a corresponding lobe of the engagement portion of the driver.

26. The method of claim 25, wherein the engagement portion of the driver comprises:
at least two lobes spaced about a perimeter of the engagement portion,
a flute on each side of each lobe of the engagement portion, the flute being recessed relative to the conical surface, and
each of the at least two lobes are spaced from each other lobe by a portion of the conical surface.

27. The method of claim 24, wherein the tapered sidewall of the implant and the tapered conical surface of the engagement portion of the driver have substantially a same angle of taper such that inserting the engagement portion of the driver into the socket of a bone implant comprises contacting the tapered sidewall along an entirety of its length with a portion of the tapered conical surface of the engagement portion of the driver.

28. The method of claim 24, wherein:
the engagement portion has a number of lobes less than a number of the plurality of grooves;
the number of the plurality of grooves of the socket is at least twice the number of lobes on the engagement portion of the driver; and
inserting the engagement portion of the driver into the socket of the bone implant comprises engaging half or less of the plurality of grooves of the implant socket with a corresponding lobe of the engagement portion of the driver.

29. The method of claim 24, wherein:
the socket of the implant is a hexalobular socket having six grooves and six protrusions; and
the engagement portion of the driver has three lobes spaced substantially equidistant about a perimeter of the engagement portion.

30. The method of claim 29, wherein the driver is a first driver, and further comprising:
disengaging the first driver from the socket of the implant; and
inserting a hexalobular driver into the socket, the hexalobular driver comprising an engagement portion defining a straight sidewall and six lobes, wherein inserting the hexalobular driver into the socket comprises engaging the six lobes with corresponding ones of the six grooves of the socket.

31. A minimally invasive provisional bone fixation technique, the technique comprising:
making an incision through a skin of a patient and retracting the skin along the incision to expose at least a portion of a first bone;
inserting a bone implant detachably engaged to a driver through the first bone and into a second bone across a joint between the first bone and the second bone;
after inserting the bone implant, detaching the driver from a proximal end of the bone implant and repositioning the skin of the patient at least partially over the proximal end of the bone implant, thereby exposing a different portion of the joint through the incision;
attaching a bone fixation device across the different portion of the joint exposed by repositioning of the skin;
after attaching the bone fixation device, performing a second repositioning of the skin to at least partially cover the bone fixation device and re-expose the proximal end of the bone implant through incision; and
engaging the driver with the proximal end of the bone implant and removing the bone implant from the first bone and the second bone.
